(19) United States
(12) Patent Application Publication
(10) Pub. No.: US 2026/0314926 A1
(43) Pub. Date: Oct. 15, 2026

(54) OPTICAL COHERENCE TOMOGRAPHY (OCT) GUIDANCE USING COMMON PATH INTERFEROMETRY FOR OPHTHALMIC SURGERY INTRODUCTION

(71) Applicant: Alcon Inc., Fribourg (CH)

(72) Inventors: Corey Stewart, Laguna Niguel, CA (US); Keith Watanabe, Irvine, CA (US); Hongwu Ren, Irvine, CA (US); Alireza Mirsepassi, Irvine, CA (US)

(21) Appl. No.: 19/567,643

(22) Filed: Mar. 16, 2026

Related U.S. Application Data
(60) Provisional application No. 63/789,060, filed on Apr. 15, 2025.

(51) Int. Cl. A61B 3/10; A61B 3/00; A61F 9/008

(52) U.S. Cl. CPC A61B 3/102; A61B 3/0025; A61F 9/008; A61B 2505/05

(57) ABSTRACT

In certain embodiments, a surgical laser system includes a laser probe and an OCT subsystem. The laser probe includes an optical fiber and an optical tip that includes an optical element configured to emit a portion of an OCT light beam toward tissue, reflect a portion of the OCT light beam to form a reference light beam, and receive reflected light from the tissue to form a sample light beam. The OCT subsystem includes an OCT light source, OCT optical components, and OCT data acquisition components. The OCT optical components are configured to transmit the OCT light beam from the OCT light source to the optical fiber, receive the reference and sample light beams from the optical fiber, and generate interference signals based on the reference and sample light beams. The OCT data acquisition components are configured to generate image data based on the interference signals.

The present disclosure relates to surgical systems. More particularly, the present disclosure relates to surgical systems and methods that use Optical Coherence Tomography (OCT) guidance for ophthalmic surgery.

Laser light may be used to treat patients in a wide variety of surgical procedures. For example, a surgical system may use laser light to treat a patient's eye during an ophthalmic surgical procedure, such as glaucoma surgery, vitreoretinal surgery, etc. During glaucoma surgery, laser light may be used to create openings in the tissue in the anterior chamber of the patient's eye. During vitreoretinal surgery, laser light may be used to cut and remove the vitreous and other tissues in the posterior chamber of the patient's eye. Laser light may also be used in other types of ophthalmic surgical procedures, such as cataract surgery, etc.

### SUMMARY

In certain embodiments, a surgical laser system comprises a laser probe and an OCT subsystem. The laser probe comprises an optical fiber and an optical tip. The optical fiber is configured to convey an OCT light beam, a reference light beam, and a sample light beam. The optical tip comprises a distal portion of the optical fiber, and at least one optical element configured to emit a first portion of the OCT light beam toward tissue, reflect a second portion of the OCT light beam to form the reference light beam, and receive reflected light from the tissue to form the sample light beam. The OCT subsystem is coupled to the laser probe, and comprises an OCT light source, OCT optical components coupled to the OCT light source, and OCT data acquisition components coupled to the OCT optical components. The OCT light source is configured to generate the OCT light beam. The OCT optical components are configured to transmit the OCT light beam from the OCT light source to the optical fiber in the laser probe, receive the reference light beam and the sample light beam from the optical fiber in the laser probe, and generate interference signals based on the reference light beam and the sample light beam. The OCT data acquisition components are configured to generate image data based on the interference signals.

### BRIEF DESCRIPTION OF THE DRAWINGS

FIG. 1A illustrates an example surgical laser system for performing an ophthalmic surgical procedure, in accordance with certain embodiments of the present disclosure.

FIG. 1B illustrates another example surgical laser system for performing an ophthalmic surgical procedure, in accordance with certain embodiments of the present disclosure.

FIG. 2 depicts an example laser probe for a surgical laser system, in accordance with certain embodiments of the present disclosure.

FIG. 3 depicts an example OCT subsystem for a surgical laser system, in accordance with certain embodiments of the present disclosure.

FIG. 4 depicts another example OCT subsystem for a surgical laser system, in accordance with certain embodiments of the present disclosure.

FIG. 5 depicts a flow diagram describing certain functionality of the surgical laser system, in accordance with certain embodiments of the present disclosure.

### DETAILED DESCRIPTION

Embodiments of the present disclosure will now be described with reference to the figures, in which like reference numerals refer to like parts throughout.

A surgical system that uses lasers for treatment is also known as a surgical laser system. Generally, a surgical laser system includes a laser probe and a surgical console. The laser probe includes a handpiece with an optical tip (or head), an optical cable that includes one or more optical fibers, and an optical connector that is attached to, and detached from, an optical port of the surgical console. The optical cable may also include one or more electrical wires, and the optical connector and the optical port may also include one or more electrical connectors. The surgical console houses various subsystems, such as a laser subsystem, an illumination subsystem, a visualization subsystem, etc.

The laser subsystem includes a treatment light source that generates a treatment light beam, such as an infrared (IR) pulsed laser that generates an IR light beam, etc. The treatment light source is optically coupled to the laser probe via the optical port, and the surgeon guides the optical tip of the laser probe handpiece to direct the treatment light beam to the ophthalmic (ocular) tissue. The illumination subsystem includes an illumination light source that generates an illumination light beam, such as one or more continuous wave lasers, etc. The illumination light beam may include one or more colors in the visible spectrum, such as red, blue, green, white, etc., and may also be known as an aiming light beam. The illumination light source is optically coupled to the laser probe via the optical port, and the optical tip of the laser probe handpiece directs the illumination light beam to the ophthalmic tissue. The visualization subsystem may include a camera, a camera interface, and a display. The camera is located within the optical tip of the laser probe handpiece, while the camera interface is located within the surgical console. The camera interface is electrically coupled to the camera via the optical port. The camera provides images of the ophthalmic tissue that is illuminated by the illumination light beam.

In addition to the visualization subsystem, the surgical laser system may include an OCT subsystem to help guide the surgeon during ophthalmic surgery. Generally, the OCT subsystem may provide one dimensional (1D) images, two dimensional (2D) images, and/or three dimensional (3D) images of the ophthalmic tissue using interferometry. The OCT subsystem may generate a 1D image of the ophthalmic tissue by performing a single A scan at a specific location, which provides a depth profile of the ophthalmic tissue at that location. The OCT subsystem may generate a 2D image by performing multiple A scans along a lateral direction (known as a B scan), which provides a cross-sectional view of the ophthalmic tissue along the lateral direction. The OCT subsystem may generate a 3D image by performing multiple B scans across a transverse direction of an area, which provides a volumetric view of the ophthalmic tissue.

The OCT subsystem includes an OCT light source and an OCT detector that are optically coupled to the laser probe via the optical port. The OCT light source generates a coherent light beam, and the optical tip of the laser probe handpiece directs the coherent light beam toward the ophthalmic tissue. The ophthalmic tissue reflects a portion of the coherent light beam back toward the optical tip as a scattered light beam, which is received and conveyed to the OCT detector. The OCT detector measures the interference between the coherent light beam from the reference arm and the reflected light beam from the sample arm, which is directly related to the reflectivity of the ophthalmic tissue. Images of the ophthalmic tissue are generated based on the OCT detector measurements to assist the surgeon in guiding the treatment light beam.

Generally, an OCT subsystem includes a double-path interferometer that generates the interference patterns, such as a Michelson interferometer, etc. The double-path interferometer splits the coherent light beam generated by the OCT light source into two separate paths or arms, such as a sample arm and a reference arm.

The sample arm includes an optical path that uses fiber optic light transmission. The length of the sample arm optical path is fixed and extends from the optical tip of the laser probe handpiece through the handpiece, the optical cable, the optical connector, and the optical port, and then through a length of optical fiber that optically couples the optical port to the OCT detector. The sample arm includes a focusing lens that is located at the optical tip of the laser probe handpiece. The coherent light beam from the OCT light source is coupled into the sample arm optical path, and the focusing lens directs the coherent light beam from the OCT light source to the ophthalmic tissue. The focusing lens also receives the light beam that is reflected from the ophthalmic tissue, which is formed into a sample light beam that is conveyed to the OCT detector along the sample arm optical path.

The reference arm includes an optical path that uses free-space light transmission, fiber optic light transmission, or a combination of free-space and fiber optic light transmission. The length of the reference arm optical path is adjustable and extends from the OCT light source to the OCT detector. The reference arm may include an adjustable mirror, a cornercube or other component that reflects the coherent light beam from the OCT light source to the OCT detector as a reference light beam. The optical path length of the reference arm may be increased or decreased by moving the adjustable mirror along a portion of the reference arm optical path to match the optical path length of the sample arm. Alternatively, the reference arm may include a fixed mirror and a variable delay line (VDL) instead of an adjustable mirror. The reference delay line can be implemented as a reflective type with only one port acting as input and output ports, or as a transmissive type with two ports, one as input and the other as output. When the reference arm optical path length and the sample arm optical path length are matched, and the polarization states of the reference arm and the sample arm are aligned, then a good interference signal may be generated.

The sample light beam is combined with the reference light beam, and the OCT detector measures the interference signal between the sample light beam and the reference light beam, from which images of the ophthalmic tissue are generated.

When the surgeon guides the laser probe during ophthalmic surgery, this motion bends and twists the optical fiber in the optical cable that conveys the sample light beam from the optical tip of the laser probe handpiece to the optical port of the surgical console. Unfortunately, bending and twisting the optical fiber in the sample arm introduces a change in birefringence of the optical fiber in the optical cable, which changes the polarization states of the sample light beam in the sample arm. The difference in the polarization states between the reference light beam in the reference arm and the sample light beam in the sample arm will randomly change the amplitude of the optical signal measured by the OCT detector. This amplitude instability in the optical signal measured by the OCT detector degrades the quality of the images that are generated based on the OCT measurements (such as image clarity, fidelity, signal-to-noise ratio (SNR), etc.). Poor quality OCT-based images impair the guidance of the laser probe during ophthalmic surgery.

Embodiments of the present disclosure advantageously provide a common optical path for the reference arm and the sample arm that reduces or eliminates the effect from polarization differences caused by uncommon optical paths of reference and sample arms, which reduces or eliminates the amplitude instability of the optical signal measured by the OCT detector thereby improving the quality of the images that are generated based on the OCT measurements.

While described in the context of an ophthalmic surgical procedure, the systems and methods described herein may be used for any suitable medical procedure that uses laser light for treatment purposes, such as tumor removal, wound and scar treatments, dermatology procedures, etc.

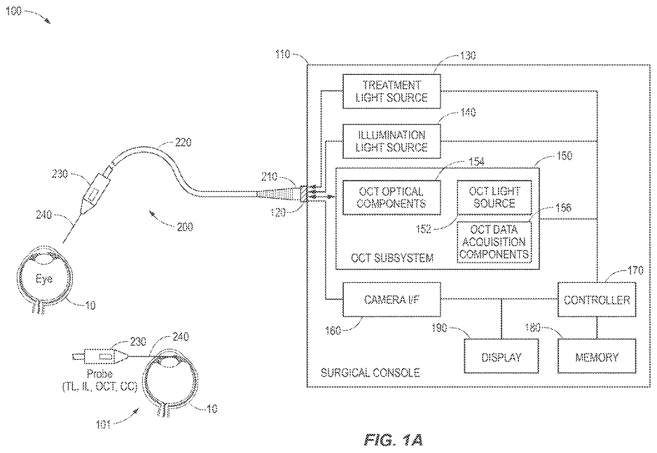

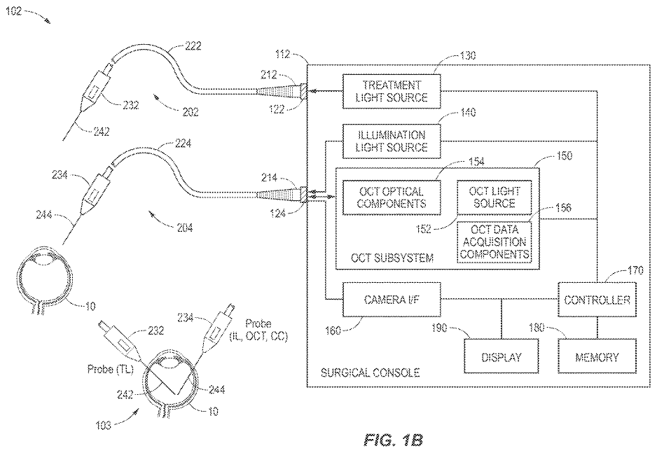

FIG. 1A illustrates an example surgical laser system 100 for performing a laser-assisted ophthalmic surgical procedure on a patient's eye 10, in accordance with certain embodiments of the present disclosure.

In certain embodiments, the surgical laser system 100 includes, among other things, a surgical console 110 and a laser probe 200. Generally, the surgical laser system 100 may be used in a variety of ophthalmic surgical procedures, such as glaucoma surgery, vitreoretinal surgery, cataract surgery, etc. In certain embodiments, the surgical laser system 100 may be configured to support a particular ophthalmic surgical procedure.

The surgical console 110 may include, among other things, an electro-optical (EO) port 120, a treatment light source 130, an illumination light source 140, an OCT subsystem 150, a camera interface (I/F) 160, a controller 170, a memory 180, and a display 190. The surgical console 110 may also include additional tools and subsystems to facilitate the performance of the ophthalmic surgical procedure. One example of a surgical console configured for performing anterior and posterior segment ophthalmic surgery is the Constellation® System available from Alcon Laboratories, Inc., Fort Worth, Texas.

The EO port 120 is configured to receive and secure the EO connector 210 of the laser probe 200. The EO port 120 is optically coupled to the treatment light source 130, the illumination light source 140, and the OCT subsystem 150 using separate optical fibers, such as a treatment optical fiber, an illumination optical fiber, etc. The EO port 120 is electrically coupled to the camera I/F 160 using electrical wires.

The treatment light source 130 is configured to generate laser light that is formed into a treatment light beam. In certain embodiments, the treatment light source 130 is a pulsed laser that generates a treatment light beam in the IR spectrum (for example, about 750 nm (nanometers) to 3000 nm) in response to control signals provided by the controller 170. The treatment light beam includes short, periodic IR laser pulses, such as 2-5 nanosecond pulses at a 1 kHz (kilohertz) pulse rate. Each IR laser pulse may have a pulse shape that is defined by the intensity of the IR laser pulse over the duration of the pulse, such as a “Gaussian” pulse shape.

The illumination light source 140 is configured to generate light that is formed into an illumination light beam. In certain embodiments, the illumination light source 140 includes three continuous wave (CW) lasers that generate laser light beams in the red, green, and blue spectrums in response to control signals provided by the controller 170. The red, green, and blue laser light beams are optically combined to form a white (or pseudo-white) illumination light beam in the visible spectrum (for example, about 380 nm to 750 nm). Other visible spectrum (color) combinations may also be used. In other embodiments, the illumination light source 140 includes three light emitting diodes (LEDs) that generate light beams in the red, green, and blue spectrums in response to control signals provided by the controller 170. The red, green, and blue light beams are optically combined to form a white (or pseudo-white) illumination light beam in the visible spectrum. In some embodiments, the illumination light source 140 includes a single CW laser or LED that generates a monochromatic light beam, such as a light beam in the red spectrum, the green spectrum, the blue spectrum, etc.

The OCT subsystem 150 may include an OCT light source 152, OCT optical components 154, and OCT data acquisition components 156. The OCT subsystem 150 uses a common optical path interferometer architecture and processes the interference signals in the Fourier or frequency domain (FD).

The OCT light source 152 is optically coupled to the OCT optical components 154 using an optical fiber and electrically coupled to the OCT data acquisition components 156 using electrical wires or cables.

In certain embodiments, the OCT subsystem 150 is a swept source OCT (SS-OCT) subsystem, and the OCT light source 152 is a swept-source laser that generates a coherent light beam that is rapidly swept (or varied) over a broad range of wavelengths over a short period of time. The light reflected from the ophthalmic tissue at each wavelength generates an interference fringe when combined with a portion of the coherent light beam reflected from the probe tip, and the interference fringes are detected and processed to generate an image (such as a 1D image, a 2D image, or a 3D image).

In other embodiments, the OCT subsystem 150 is a spectral domain OCT (SD-OCT) subsystem, and the OCT light source 152 is a broadband light source (such as a superluminescent diode) that generates a broadband light beam. The broad spectrum of wavelengths produces simultaneous reflection of light from different depths of the ophthalmic tissues. The light reflected from the ophthalmic tissue generates interference fringes when combined with a portion of the broadband light beam reflected from the probe tip, which are detected and processed to generate an image.

The OCT optical components 154 include optical and electro-optical components that guide and adjust the outgoing and incoming light beams, generate and detect interference fringes, and generate interference signals. The OCT optical components 154 are optically coupled to the EO port 120 using an optical fiber and electrically coupled to the OCT data acquisition components 156 using electrical wires or cables.

Generally, the OCT light source 152 generates an OCT light beam that is provided to the OCT optical components 154, such as a coherent light beam from a swept-source laser or a broadband light beam from a broadband light source. The laser probe 200 receives the OCT light beam from the OCT optical components 154, illuminates the ophthalmic tissue using the OCT light beam, and reflects a portion of the OCT light beam from the optical tip 240 to the OCT optical components 154 as a reference light beam. The laser probe 200 also receives the light reflected by the ophthalmic tissue, and sends the light reflected by the ophthalmic tissue from the optical tip 240 to the OCT optical components 154 as a sample light beam. The OCT optical components 154 generate an interference signal based on the reference and sample light beams and send the interference signal to the OCT data acquisition components 156.

The OCT data acquisition components 156 may include a processor, memory, and signal conditioning and processing circuitry, such as an analog-to-digital (A/D) converter, a video frame grabber card, etc. Generally, the OCT data acquisition components 156 accept the trigger and clock control signals from the OCT light source 152, process the interference signals, generate the images, and send the images to the controller 170 for further processing.

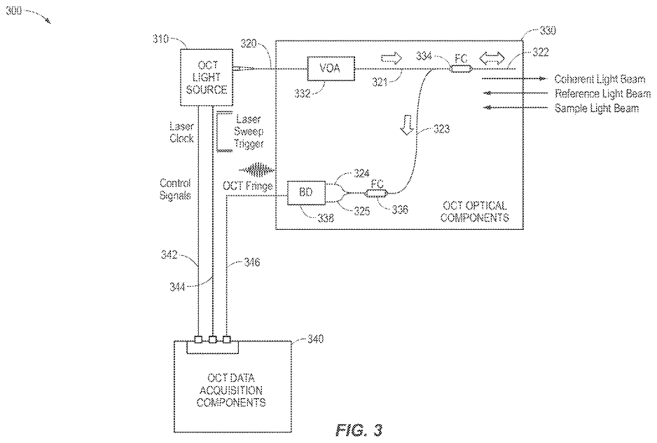

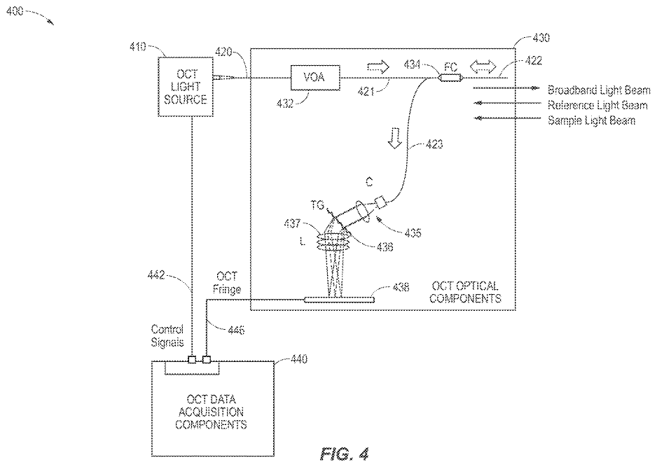

An example of an SS-OCT subsystem is described in more detail below with respect to FIG. 3, and an example of an SD-OCT subsystem is described in more detail below with respect to FIG. 4.

The controller 170 is coupled to the treatment light source 130, the illumination light source 140, the OCT subsystem 150, the camera I/F 160, the memory 180, and the display 190. The controller 170 may be a microcontroller, a microprocessor, a programmable logic controller (PLC), a field programmable gate array (FPGA), an application specific integrated circuit (ASIC), etc.

Generally, the controller 170 is configured to control the operation of the treatment light source 130 through various settings (such as pulse width or duration, pulse frequency or period, wavelength, power or intensity, spot size, etc.), and to generate control signals for the treatment light source 130. The controller 170 is also configured to control the operation of the illumination light source 140 through various settings (such as power or intensity, wavelength, etc.), and to generate control signals for the illumination light source 140. The controller 170 is also configured to control the operation of the OCT subsystem 150, to receive and process image data from the OCT subsystem 150, and to present the image data on the display 190. The controller 170 is also configured to control to receive and process image data from the camera I/F 160 and present the image data on the display 190.

The laser probe 200 includes, among other things, the EO connector 210, an EO cable 220, and a handpiece 230 including an optical tip 240. The distal end of the EO cable 220 may be directly attached to the handpiece 230; alternatively, the distal end of the EO cable 220 may be removably coupled to the handpiece 230 using an EO connector. The proximal end of the EO cable 220 includes the EO connector 210, which may be attached to, and detached from, the EO port 120. Several optical fibers and electrical wires within the EO cable 220 and the handpiece 230 couple the EO connector 210 to the optical tip 240.

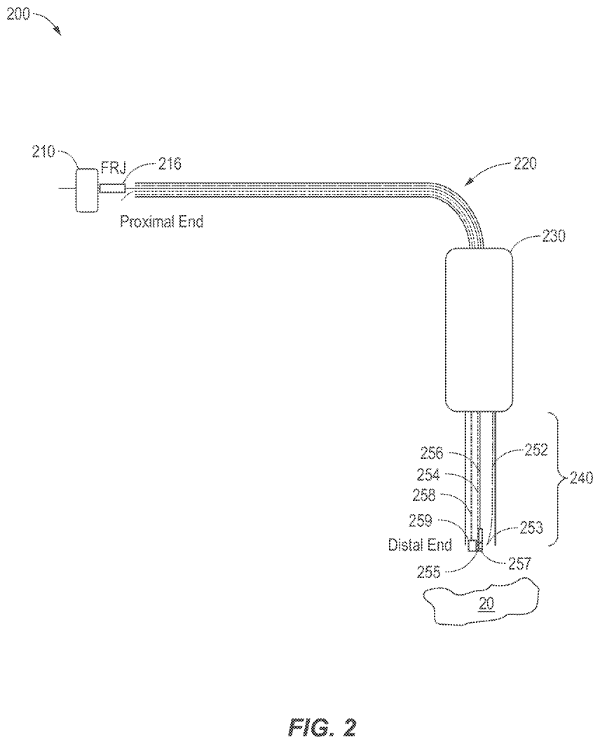

FIG. 2 depicts an example laser probe 200 for a surgical laser system 100, in accordance with certain embodiments of the present disclosure.

In certain embodiments, the EO cable 220 and the handpiece 230 include optical fibers 252, 254, 256. In certain embodiments, the optical fibers may include single-core optical fibers (SCFs), multi-core optical fibers (MCFs), such as GeO2 with tapped sapphire cable, a combination of single-core and multi-core fibers, etc. Other fiber optical materials may also be used, such as Ti:Sapphire, Y3Al5O12 (YAG), Ho: YAG, Yb:YAG, Nd:YAG, Er:YAG, Ce:YAG, Cr:YAG, or ZrF4-BaF2-LaF3-AlF3-NaF (ZBLAN), etc.

The optical fiber 252 conveys the treatment light beam from the EO connector 210 to the optical tip 240 and may have an optical element 253 (such as a focusing lens, a lensed tip, etc.) attached to, or formed in, the distal end.

The optical fiber 254 conveys the illumination light beam from the EO connector 210 to the optical tip 240 and may have an optical element 255 (such as focusing lens, a lensed tip, etc.) attached to, or formed in, the distal end.

The optical fiber 256 conveys the OCT light beam from the EO connector 210 to the optical tip 240 and conveys the reference light beam and the sample light beam from the optical tip 240 to the EO connector 210. The optical fiber 256 may have at least one optical element 257 attached to, or formed in, the distal end.

For example, the optical element 257 may include a polished angled tip that is coated with an anti-reflection coating (such as 5% to 10% reflectivity) to customize the back-reflection from the distal end of the optical fiber 256. The back-reflected light from the distal end forms the reference light beam, while the angled polished tip emits the OCT light beam towards the ophthalmic tissue 20 and receives the reflected light from the ophthalmic tissue 20, which is formed into the sample light beam. In another example, the optical element 257 may include a focusing lens (such as a gradient-index or GRIN lens) that has an anti-reflective coating to customize the back-reflection. The focusing lens may be attached to the end of the optical fiber 256 using an optical adhesive that has a refractive index that matches the refractive index of the optical fiber 256. In a further example, a prism and a spacer may be attached between the end of the optical fiber 256 and the focusing lens using the optical adhesive.

In some embodiments, a 2×1 fiber coupler may combine the treatment light beam transmitted in one optical fiber and the illumination light beam transmitted in another optical fiber into a single optical fiber that is coupled to the EO port 120. The optical fiber 254 then conveys the treatment light beam and the illumination light beam from the EO connector 210 to the optical tip 240, and the optical fiber 252 is not needed.

In other embodiments, a 2×1 fiber coupler combines the illumination light beam transmitted in one optical fiber and the OCT light beam transmitted in another optical fiber into a single optical fiber that is coupled to the EO port 120. The optical fiber 256 then conveys the illumination light beam and the OCT light beam from the EO connector 210 to the optical tip 240, and the optical fiber 254 is not needed.

In certain embodiments, the OCT subsystem 150 may generate a 1D image of the ophthalmic tissue 20 using an A scan. The 1D image may be used to determine the distance from the optical tip 240 to the ophthalmic tissue 20. The A scan does not require displacement of the OCT light beam along lateral or transverse directions.

In other embodiments, the OCT subsystem 150 may generate a 2D image of the ophthalmic tissue 20 using a B scan, which performs multiple A scans along a lateral direction. The B scan requires incremental displacement of the OCT light beam along the lateral direction for each A scan.

In one example, a fiber rotary joint (FRJ) 216 may be coupled to the EO connector 210 and to the proximal end of the optical fiber 256. A motor drive coupled to the FRJ 216 rotates the optical fiber 256 and the optical element 257 between a positive displacement angle (such as +45°) and a negative displacement angle (such as −45°) in a lateral direction to perform the A scans that comprise the B scan. The motor drive may be coupled to, and controlled by, the OCT data acquisition components 156. In another example, the optical element 257 may also include a deflection element, such as an articulated PZT (Lead Zirconate Titanate) or MEMS (Micro-Electro-Mechanical Systems) mirror, an articulated prism, etc., that moves the OCT light beam between a positive displacement angle (such as +45°) and a negative displacement angle (such as −45°) in a lateral direction to perform the A scans that comprise the B scan. The deflection element may be coupled to, and controlled by, the OCT data acquisition components 156. Other displacement angles may also be used, such as ±30°, ±15°, etc.

In some embodiments, the OCT subsystem 150 may generate a 3D image of the ophthalmic tissue 20 by displacing the deflection element along a transverse direction to perform the B scans that comprise the volumetric scan.

The EO cable 220 and the handpiece 230 also include a number of electrical wires 258. The electrical wires 258 may include, among other things, power wires to convey positive and negative DC voltages from the EO connector 210 to a camera 259 located in the optical tip 240, one or more signal wires to convey video signals from the camera 259 to the EO connector 210, etc. The illumination light beam illuminates the ophthalmic tissue 20 for the camera 259. In some embodiments, the camera may be located in the camera I/F 160, and the EO cable 220 and the handpiece 230 may include an optical fiber bundle that conveys an optical image from the optical tip 240 to the EO connector 210. The EO port 120 may be optically coupled to the camera I/F 160 using an optical fiber bundle.

Referring back to FIG. 1A, the surgeon may activate and deactivate the treatment light source 130 using a mechanical or electrical control, such as a foot pedal, a switch on the handpiece 230, etc. The mechanical or electrical control may be coupled to the controller 170 or directly to the treatment light source 130. In one example, an electrical cable may couple the foot pedal to the controller 170. In another example, one or more electrical wires located within the EO cable 220 and handpiece 230 may couple the switch to the EO connector 210.

The laser probe 200 may be used during glaucoma surgery 101 in the anterior chamber of the eye 10 to treat different areas in the trabecular meshwork spatially adjacent to the Schlemm's canal. During glaucoma surgery 101, the optical tip 240 is inserted into the anterior chamber of the eye 10, and the surgeon directs the treatment light beam to different areas in the trabecular meshwork spatially adjacent to the Schlemm's canal, guided by the images generated by OCT subsystem 150 and the video signals generated by the camera in the optical tip 240 that are processed by the camera I/F 160 and presented on the display 190.

FIG. 1B illustrates another example surgical laser system 102 for performing a laser-assisted ophthalmic surgical procedure on a patient's eye 10, in accordance with certain embodiments of the present disclosure.

In certain embodiments, the surgical laser system 102 includes, among other things, a surgical console 112, a treatment laser probe 202, and a visualization laser probe 204. Generally, the components of the laser probe 200 described above are divided between the treatment laser probe 202 and the visualization laser probe 204, and the EO port 120 is replaced by an optical port 122 and an EO port 124. In certain embodiments, the treatment laser probe 202 includes the components related to the treatment light source 130, while the visualization laser probe 204 includes the components related to the illumination light source 140, the OCT subsystem 150, and the camera and camera I/F 160. The surgical laser system 102 may also be used in a variety of ophthalmic surgical procedures, such as glaucoma surgery, vitreoretinal surgery, cataract surgery, etc.

The surgical console 112 may include, among other things, the treatment light source 130, the illumination light source 140, the OCT subsystem 150, the camera I/F 160, the controller 170, the memory 180, and the display 190, as described above with respect to the surgical console 110, as well as the optical port 122 and the EO port 124. The surgical console 112 may also include additional tools and subsystems to facilitate the performance of the ophthalmic surgical procedure.

The optical port 122 is configured to receive and secure the optical connector 212 of the treatment laser probe 202. The optical port 122 is optically coupled to the treatment light source 130 using an optical fiber.

The EO port 124 is configured to receive and secure the EO connector 214 of the visualization laser probe 204. The EO port 124 is optically coupled to the illumination light source 140 and the OCT subsystem 150 using separate optical fibers and is electrically coupled to the camera I/F 160 using electrical wires.

The treatment laser probe 202 includes, among other things, the optical connector 212, an optical cable 222, and a handpiece 232 including an optical tip 242. The distal end of the optical cable 222 may be directly attached to the handpiece 232; alternatively, the distal end of the optical cable 222 may be removably coupled to the handpiece 232 using an optical connector. The proximal end of the optical cable 222 includes the optical connector 212, which may be attached to, and detached from, the optical port 122.

An optical fiber within the optical cable 222 and the handpiece 232 couples the optical connector 212 to the optical tip 242 and conveys the treatment light beam from the optical connector 212 to the optical tip 242, as discussed above with respect to the optical fiber 252. The optical fiber may have an optical element (such as a focusing lens, a lensed tip, etc.) attached to, or formed in, the distal end, as described above with respect to the optical element 253.

The visualization laser probe 204 includes, among other things, the EO connector 214, an EO cable 224, and a handpiece 234 including an optical tip 244. The distal end of the EO cable 224 may be directly attached to the handpiece 234; alternatively, the distal end of the EO cable 224 may be removably coupled to the handpiece 234 using an EO connector. The proximal end of the EO cable 224 includes the EO connector 214, which may be attached to, and detached from, the EO port 124.

Several optical fibers and electrical wires within the EO cable 224 and handpiece 234 couple the EO connector 214 to the optical tip 244.

One optical fiber conveys the illumination light beam from the EO connector 214 to the optical tip 244, as discussed above with respect to the optical fiber 254. This optical fiber may have an optical element (such as a focusing lens, a lensed tip, etc.) attached to, or formed in, the distal end, as described above with respect to the optical element 255.

Another optical fiber conveys the OCT light beam from the EO connector 214 to the optical tip 244 and conveys the reference light beam and the sample light beam from the optical tip 244 to the EO connector 214. This optical fiber may have one or more optical elements attached to, or formed in, the distal end, as described above with respect to the optical element 257.

The EO cable 224 and the handpiece 234 also include a number of electrical wires, as described above with respect to electrical wires 258. The electrical wires may include, among other things, power wires to convey positive and negative DC voltages from the EO connector 214 to a camera located in the optical tip 244, one or more signal wires to convey video signals from the camera to the EO connector 214, etc. The illumination light beam illuminates the ophthalmic tissue for the camera. In some embodiments, the camera may be located in the camera I/F 160, and the EO cable 224 and the handpiece 234 may include an optical fiber bundle that conveys an optical image from the optical tip 244 to the EO connector 214. The EO port 124 may be optically coupled to the camera I/F 160 using an optical fiber bundle.

The treatment laser probe 202 and the visualization laser probe 204 may be used during vitreoretinal surgery 103 in the posterior chamber of the eye 10. During vitreoretinal surgery 103, the optical tip 242 of the treatment laser probe 202 and the optical tip 244 of the visualization laser probe 204 are inserted into the posterior chamber of the eye 10, and the surgeon independently directs the treatment light beam emitted by the optical tip 242 and the illumination light beam emitted by the optical tip 244 and toward the ophthalmic tissue, guided by the images generated by OCT subsystem 150 and the images generated by the camera in the optical tip 244 that are presented on the display 190. The laser probe 200 of the surgical laser system 100 (FIG. 1A) may also be used for vitreoretinal surgery 103 in the posterior chamber of the eye 10.

In certain embodiments, the treatment light source 130, the optical port 122, and the treatment laser probe 202 may be replaced by an ultrasonic source, an electrical port, and an ultrasonic probe, respectively. The electrical port may be configured to receive and secure the electrical connector of the ultrasonic probe and is electrically coupled to the ultrasonic source using an electrical cable. The ultrasonic probe may include the electrical connector, an electrical cable, and a handpiece with an ultrasonic tip. The ultrasonic probe and the visualization laser probe 204 may be cooperatively used during vitreoretinal surgery 103 in the posterior chamber of the eye 10.

As discussed above, in certain embodiments, the OCT subsystem 150 may be an SS-OCT subsystem.

FIG. 3 depicts an example SS-OCT subsystem 300 for the surgical laser systems 100, 102, in accordance with certain embodiments of the present disclosure.

In certain embodiments, the SS-OCT subsystem 300 includes, among other things, an OCT light source 310, OCT optical components 330, and OCT data acquisition components 340. The SS-OCT subsystem 300 uses a common optical path interferometer architecture and processes the interference signals in the Fourier or frequency domain (FD).

The OCT light source 310 is a swept-source laser that generates a coherent light beam that is rapidly swept (or varied) over a broad range of wavelengths over a short period of time, as discussed above.

The OCT optical components 330 include optical and electro-optical components including a variable optical attenuator (VOA) 332, a fiber coupler (FC) 334, a fiber coupler 336, and a balance detector (BD) 338, which are optically coupled using optical fibers.

The OCT light source 310 is coupled to the input port of the VOA 332 via the optical fiber 320. The VOA 332 is a passive optical device that reduces the optical power of the coherent light beam to prevent signal saturation during acquisition. The amount of attenuation provided by the VOA 332 may be configurable. The output port of the VOA 332 is coupled to an input port of the fiber coupler 334 via optical fiber 321.

The fiber coupler 334 is a “2×1” fiber coupler with two input ports and one output port. One input port receives the coherent light beam from the VOA 332 and passes the coherent light beam to the output port, which is coupled to the EO port 120 of the surgical console 110 (or the EO port 124 of the surgical console 112) via the optical fiber 322. When the EO connector 210 of the laser probe 200 is attached to the EO port 120, the optical fiber 322 is optically coupled to the optical fiber 256 of the laser probe 200.

In addition to transmitting the coherent light beam to the EO port 120, the output port of the fiber coupler 334 also receives the reference and sample light beams from the EO port 120. The fiber coupler 334 passes the reference and sample light beams to the second input port (which functions as an output port), which is coupled to the input port of the fiber coupler 336 via the optical fiber 323. In other words, the fiber coupler 334 combines the coherent reference and sample light beams travelling in the same optical path, after the sample light beam reflected back into the optical tip.

The common optical path for the reference and sample arms of the SS-OCT subsystem 300 begins at the optical element 257, includes the optical fiber 256 and the optical fiber 322, and ends at the fiber coupler 334. A detection arm begins at the fiber coupler 334, includes the optical fiber 323, the fiber coupler 336, and the optical fibers 324, 325, and ends at the balanced detector 338. The common optical path for the reference and sample arms advantageously reduces the polarization effect to a significant degree, which significantly improves the stability of the amplitude of the detected interference signal.

The fiber coupler 336 is in other words “1×2” (50/50) fiber coupler with one input port and two output ports. The input port receives the reference and sample light beams from the optical fiber 323 and divides the reference and sample light beams into two equal light beams, which are separately provided to the input ports of the balanced detector 338 via the optical fibers 324 and 325.

The balanced detector 338 includes a photodetector coupled to each input port, and signal processing components coupled to the photodetectors. Each photodetector detects the intensity of the respective interference fringes of input light beams and generates an electrical signal (such as a voltage signal) that is proportional to the intensity. The signal processing components electronically subtract the electrical signals from the photodetectors to generate a difference signal that is cleaner and less noisy than the electrical signals.

In one example, the signal processing components may include a differential amplifier circuit, which applies the electrical current signals from the photodetectors to the inverting and non-inverting inputs of a transimpedance amplifier (a type of op-amp) to convert current signals to voltage signals. The output signal (output voltage) of the op-amp represents the difference between the electrical signals from the photodetectors, which is provided to the OCT data acquisition components 340 as an interference signal via the electrical cable 346.

The OCT data acquisition components 340 may include a processor, memory, and signal conditioning and processing circuitry, such as an analog-to-digital (A/D) converter, FPGA etc. Generally, the OCT data acquisition components 340 accept the control signals from the OCT light source 310, process the interference signals from the balanced detector 338, generate images based on the processed interference signals, and send the images to the controller 170 for further processing. For example, the OCT data acquisition components 340 may digitize the interference signals and perform fast Fourier transforms (FFTs) on the digitized interference signals to generate A scan intensity profiles for the images.

The control signals for the OCT light source 310 may include a laser clock signal conveyed via the electrical cable 342, and a laser sweep signal conveyed via the electrical cable 344. The overall operation of the SS-OCT subsystem 300 is controlled by controller 170, which sends control signals to the OCT data acquisition components 340 over electrical wires or cables.

As discussed above, in certain embodiments, the OCT subsystem 150 may be an SD-OCT subsystem.

FIG. 4 depicts an example SD-OCT subsystem 400 for the surgical laser systems 100, 102, in accordance with certain embodiments of the present disclosure.

In certain embodiments, the SD-OCT subsystem 400 includes, among other things, an OCT light source 410, OCT optical components 430, and OCT data acquisition components 440. The SD-OCT subsystem 400 uses a common optical path interferometer architecture and processes the interference signals in the Fourier or frequency domain (FD).

The OCT light source 410 is a broadband light source (such as a superluminescent diode) that generates a broadband light beam, as described above.

The OCT optical components 430 include optical and electro-optical components including a variable optical attenuator (VOA) 432, and a fiber coupler 434, which are optically coupled using optical fibers. The OCT optical components 430 also include several free space optical components, such as a collimator (C) 435, a transmission grating (TG) 436, an imaging lens (L) 437, and an image capture device 438, such as a line array charge-coupled device (CCD) with 1024 pixels, 2048 pixels, etc., a complementary metal-oxide semiconductor (CMOS) camera, etc.

The OCT light source 410 is coupled to the input port of the VOA 432 via the optical fiber 420. The VOA 432 is a passive optical device that reduces the optical power of the broadband light beam to prevent signal saturation during acquisition. The amount of attenuation provided by the VOA 432 may be configurable. The output port of the VOA 432 is coupled to an input port of the fiber coupler 434 via optical fiber 421.

The fiber coupler 434 is a “2×1” fiber coupler with two input ports and one output port. One input port receives the broadband light beam from the VOA 432 and passes the broadband light beam to the output port, which is coupled to the EO port 120 of the surgical console 110 (or the EO port 124 of the surgical console 112) via the optical fiber 422. When the EO connector 210 of the laser probe 200 is attached to the EO port 120, the optical fiber 422 is optically coupled to the optical fiber 256 of the laser probe 200.

In addition to transmitting the broadband light beam to the EO port 120, the output port of the fiber coupler 434 also receives the reference and sample light beams from the EO port 120. The fiber coupler 434 passes the reference and sample light beams to the second input port (which functions as an output port), which is coupled to the collimator 435 via the optical fiber 423. In other words, the fiber coupler 434 combines the broadband reference and sample light beams travelling in the same optical path, after the sample light beam reflected back into the optical tip.

The common optical path for the reference and sample arms of the SD-OCT subsystem 400 begins at the optical element 257, includes the optical fiber 256 and the optical fiber 422, and ends at the fiber coupler 434. The optical path for the detection arm begins at the fiber coupler 434, includes the optical fiber 423, the collimator 435, the transmission grating 436, and the imaging lens 437, and ends at the image capture device 438. The common optical path for the reference and sample arms advantageously reduces the polarization effect to a significant degree, which significantly improves the stability of the amplitude of the detected interference signal.

The collimator 435 has an input port that is coupled to the optical fiber 423, an aperture, and a lens. The reference and sample light beams are emitted from the aperture as a free space divergent light beam. The free space divergent light beam is directed onto a lens, which focuses the free space divergent light beam into a free space collimated light beam. The lens is positioned at a particular distance relative to the aperture to provide the proper collimation. Generally, the aperture and the lens are contained within a housing, and the collimator 435 may be adjustable to vary the free space collimated light beam for the best collimation.

The transmission grating 436 is a type of diffraction grating in which the incident light beam passes through the grating structure (rather than reflecting off of it) and is diffracted into multiple light beams based on wavelength. The transmission grating 436 receives the free space collimated light beam, which is diffracted into a number of separate light beams.

The imaging lens 437 includes one or more lenses that focuses the light beams generated by the transmission grating 436 onto the image capture device 438.

The image capture device 438 converts the light beams for each wavelength region into electrical signals, which are provided to the OCT data acquisition components 440 as interference signals via the electrical cable 446.

The OCT data acquisition components 440 may include a processor, memory, and signal conditioning and processing circuitry, such as an analog-to-digital (A/D) converter, a video frame grabber card, etc. Generally, the OCT data acquisition components 440 accept the control signals from the OCT light source 410, process the interference signals from the image capture device 438, generate images based on the processed interference signals, and send the images to the controller 170 for further processing. For example, the OCT data acquisition components 440 may calibrate the interference signals to the wavenumber domain before performing the background subtractions and FFTs to obtain the OCT A scan intensity profiles. The OCT A scan intensity profiles may be remapped to a logarithmic scale for display. In certain embodiments, the OCT data acquisition components digitize the raw fringe signals only and leave other signal processing to controller 170.

The control signals from the OCT light source 410 may be conveyed via the electrical cable 442. The overall operation of the SD-OCT subsystem 400 is controlled by controller 170, which sends control signals to the OCT data acquisition components 440 over electrical wires or cables.

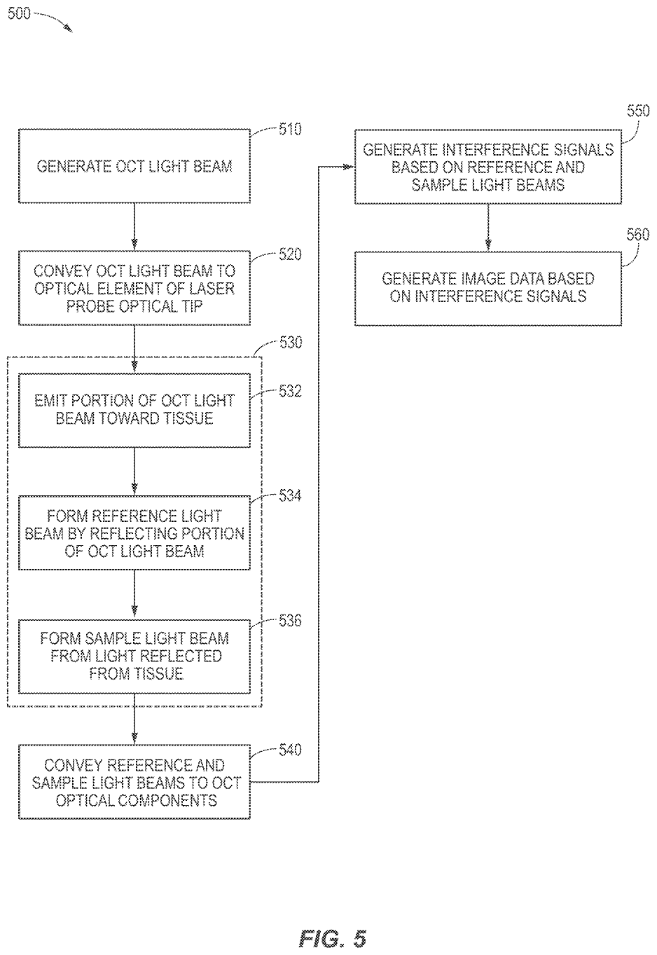

FIG. 5 depicts a flow diagram 500 describing certain functionality of the surgical laser system 100, 102 in accordance with certain embodiments of the present disclosure.

At block 510, an OCT light beam is generated by an OCT light source 152, as described above with respect to FIGS. 1A, 1B. In certain embodiments, the OCT light source 152 is OCT light source 310, such as a swept-source laser described above with respect to FIG. 3. In other embodiments, the OCT light source 152 is OCT light source 410, such as a broadband light source described above with respect to FIG. 4.

At block 520, the OCT light beam is conveyed to an optical element 257 of the optical tip 240 of the laser probe 200 by an optical fiber 256, as described above with respect to FIG. 2.

Block 530 is performed by the optical element 257, and includes blocks 532, 534, and 536.

At block 532, a portion of the OCT light beam is emitted toward tissue 20, as described above with respect to FIG. 2.

At block 534, a reference light beam is formed by reflecting a portion of the OCT light beam.

At block 536, a sample light beam is formed from light reflected from the tissue 20.

At block 540, the reference light beam and the sample light beam are conveyed, by the optical fiber 256, from the optical element 257 to an OCT optical components 154. In certain embodiments, the OCT optical components 154 are the OCT optical components 330 described above with respect to FIG. 3. In other embodiments, the OCT optical components 154 are the OCT optical components 430 described above with respect to FIG. 4.

At block 550, interference signals are generated, by the OCT optical components 154, based on the reference light beam and the sample light beam. In certain embodiments, the OCT optical components 154 are the OCT optical components 330 described above with respect to FIG. 3. In other embodiments, the OCT optical components 154 are the OCT optical components 430 described above with respect to FIG. 4.

At block 560, image data are generated, by the OCT data acquisition components 156, based on the interference signals. In certain embodiments, the OCT data acquisition components 156 are the OCT data acquisition components 340 described above with respect to FIG. 3. In other embodiments, the OCT data acquisition components 156 are the OCT data acquisition components 440 described above with respect to FIG. 4.

### Example Embodiments

In certain embodiments, a surgical laser system comprises a laser probe and an OCT subsystem. The laser probe comprises an optical fiber and an optical tip. The optical fiber is configured to convey an OCT light beam, a reference light beam, and a sample light beam. The optical tip comprises a distal portion of the optical fiber, and at least one optical element configured to emit a first portion of the OCT light beam toward tissue, reflect a second portion of the OCT light beam to form the reference light beam, and receive reflected light from the tissue to form the sample light beam. The OCT subsystem is coupled to the laser probe, and comprises an OCT light source, OCT optical components coupled to the OCT light source, and OCT data acquisition components coupled to the OCT optical components. The OCT light source is configured to generate the OCT light beam. The OCT optical components are configured to transmit the OCT light beam from the OCT light source to the optical fiber in the laser probe, receive the reference light beam and the sample light beam from the optical fiber in the laser probe, and generate interference signals based on the reference light beam and the sample light beam. The OCT data acquisition components are configured to generate image data based on the interference signals.

In certain embodiments, a method for a surgical laser system comprises generating, by an OCT light source, an OCT light beam; conveying, by an optical fiber, the OCT light beam to an optical element of a laser probe optical tip; emitting, by the optical element, a first portion of the OCT light beam toward tissue; forming, by the optical element, a reference light beam by reflecting a second portion of the OCT light beam; forming, by the optical element, a sample light beam from light reflected from the tissue; conveying, by the optical fiber, the reference light beam and the sample light beam from the optical element to OCT optical components; generating, by the OCT optical components, interference signals based on the reference light beam and the sample light beam; and generating, by OCT data acquisition components, image data based on the interference signals.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A surgical laser system, comprising:
a laser probe, comprising:
an optical fiber configured to convey an optical coherence tomography (OCT) light beam, a reference light beam, and a sample light beam, and
an optical tip comprising:
a distal portion of the optical fiber, and
at least one optical element configured to:
emit a first portion of the OCT light beam toward tissue,
reflect a second portion of the OCT light beam to form the reference light beam, and
receive reflected light from the tissue to form the sample light beam; and
an OCT subsystem coupled to the laser probe, the OCT subsystem comprising:
an OCT light source configured to generate the OCT light beam,
OCT optical components coupled to the OCT light source, the OCT optical components configured to:
transmit the OCT light beam from the OCT light source to the optical fiber in the laser probe,
receive the reference light beam and the sample light beam from the optical fiber in the laser probe, and
generate interference signals based on the reference light beam and the sample light beam, and
OCT data acquisition components coupled to the OCT optical components, the OCT data acquisition components configured to generate image data based on the interference signals.

2. The surgical laser system of claim 1, wherein the reference light beam and the sample light beam are conveyed from the optical element to the OCT optical components along a common optical path.

3. The surgical laser system of claim 2, wherein the optical element includes an anti-reflection coating that is configured to reflect between 5% and 10% of the OCT light beam.

4. The surgical laser system of claim 3, wherein the optical element comprises a polished angled tip that is formed in the distal portion of the optical fiber.

5. The surgical laser system of claim 3, wherein the optical element comprises a focusing lens that is attached to an end of the distal portion of the optical fiber using an optical adhesive that has a refractive index that matches a refractive index of the optical fiber.

6. The surgical laser system of claim 5, wherein the optical element further comprises a prism and a spacer that are disposed between the end of the distal portion of the optical fiber and the focusing lens and attached thereto using the optical adhesive.

7. The surgical laser system of claim 2, wherein the OCT optical components comprise:
a variable optical attenuator (VOA) coupled to the OCT light source; and
a 2×1 fiber coupler, coupled to the VOA and the optical fiber in the laser probe, the 2×1 fiber coupler configured to:
transmit the OCT light beam from the OCT light source to the optical fiber in the laser probe, and
receive the reference light beam and the sample light beam from the optical fiber in the laser probe.

8. The surgical laser system of claim 7, wherein:
the OCT light source is a swept-source laser; and
the OCT optical components further comprise:
a 1×2 fiber coupler coupled to the 2×1 fiber coupler, the 1×2 fiber coupler configured to receive the reference light beam and the sample light beam from the 2×1 fiber coupler, and divide the reference light beam and the sample light beam into two equal light beams, and
a balanced detector coupled to the 1×2 fiber coupler, the balanced detector configured to receive the equal light beams from the 1×2 fiber coupler, and generate an interference signal, and provide the interference signal to the OCT data acquisition components.

9. The surgical laser system of claim 7, wherein:
the OCT light source is a broadband light source; and
the OCT optical components further comprise:
a collimator coupled to the 2×1 fiber coupler, the collimator configured to receive the reference light beam and the sample light beam from the 2×1 fiber coupler, and generate a free space collimated light beam,
a transmission grating configured to receive the free space collimated light beam, and diffract the free space collimated light beam into multiple light beams based on wavelength, and
an imaging lens comprising one or more lenses that focuses the multiple light beams generated by the transmission grating onto an image capture device,
wherein the image capture device is configured to generate the interference signals, and provide the interference signals to the OCT data acquisition components.

10. The surgical laser system of claim 1, wherein the image data is an A scan providing a depth profile of the tissue at a particular location.

11. The surgical laser system of claim 1, wherein the image data is a B scan providing a cross-sectional view of the tissue along a lateral direction.

12. The surgical laser system of claim 1, further comprising:
a treatment light source configured to generate a treatment light beam; and
an illumination light source configured to generate an illumination light beam,
wherein the laser probe further comprises:
a treatment optical fiber coupled to the treatment light source,
an illumination optical fiber coupled to the illumination light source,
wherein the optical tip of the laser probe further comprises:
a distal portion of the treatment optical fiber configured to emit the treatment light beam toward the tissue, and
a distal portion of the illumination optical fiber configured to emit the illumination light beam toward the tissue.

13. The surgical laser system of claim 12, further comprising:
a camera interface configured to process video signals,
wherein the laser probe further comprises:
a camera located in the optical tip, the camera configured to generate the video signals, and
one or more electrical cables coupled to the camera and the camera interface.

14. The surgical laser system of claim 12, further comprising:
a camera interface configured to process video signals, the camera interface comprising a camera configured to generate the video signals,
wherein the laser probe further comprises an optical fiber bundle coupled to the camera interface.

15. A method for a surgical laser system, comprising:
generating, by an OCT light source, an OCT light beam;
conveying, by an optical fiber, the OCT light beam to an optical element of a laser probe optical tip;
emitting, by the optical element, a first portion of the OCT light beam toward tissue;
forming, by the optical element, a reference light beam by reflecting a second portion of the OCT light beam;
forming, by the optical element, a sample light beam from light reflected from the tissue;
conveying, by the optical fiber, the reference light beam and the sample light beam from the optical element to OCT optical components;
generating, by the OCT optical components, interference signals based on the reference light beam and the sample light beam; and
generating, by OCT data acquisition components, image data based on the interference signals.

16. The method of claim 15, wherein the reference light beam and the sample light beam are conveyed from the optical element to the OCT optical components along a common optical path.

17. The method of claim 16, wherein the optical element includes an anti-reflection coating that is configured to reflect between 5% and 10% of the OCT light beam.

18. The method of claim 17, wherein the optical element comprises a polished angled tip that is formed in a distal portion of the optical fiber.

19. The method of claim 17, wherein the optical element comprises a focusing lens that is attached to an end of a distal portion of the optical fiber using an optical adhesive that has a refractive index that matches a refractive index of the optical fiber.

20. The method of claim 15, wherein the OCT light source is a swept-source laser or a broadband light source.
